(19) United States
(12) Patent Application Publication
(10) Pub. No.: US 2025/0359739 A1
(43) Pub. Date: Nov. 27, 2025

(54) ENDOSCOPE

(71) Applicant: FUJIFILM Corporation, Tokyo (JP)

(72) Inventor: Yuto SHIMOYAMA, Ashigarakami-gun (JP)

(73) Assignee: FUJIFILM Corporation, Tokyo (JP)

(21) Appl. No.: 19/214,100

(22) Filed: May 21, 2025

(30) Foreign Application Priority Data
May 24, 2024 (JP) 2024-085063

(51) Int. Cl. A61B 1/005

(52) U.S. Cl. CPC A61B 1/0055; A61B 1/0052

(57) ABSTRACT

An endoscope includes: an insertion part including a distal end part and a bendable part provided on a proximal end side of the distal end part; and a bending operation part capable of performing a bending operation on the bendable part, the bendable part includes a first region and a second region between the first region and the distal end part, the first region is bent in one direction with a first curvature radius in accordance with an operation of the bending operation part, the second region is bent in the one direction with a second curvature radius less than the first curvature radius in accordance with an operation of the bending operation part, and a length of the second region in a longitudinal axis direction of the insertion part is equal to or less than a length of the first region in the longitudinal axis direction.

### CROSS-REFERENCE TO RELATED APPLICATION

The present application claims priority under 35 U.S.C. § 119 to Japanese Patent Application No. 2024-085063, filed on May 24, 2024. The above application is hereby expressly incorporated by reference, in its entirety, into the present application.

### BACKGROUND OF THE INVENTION

### 1. Field of the Invention

The present invention relates to an endoscope.

### 2. Description of the Related Art

JP2005-334050A, JP1990-271817A (JP-H02-271817A), JP1981-124401A (JP-S56-124401A), and JP1999-155806A (JP-H11-155806A) disclose an endoscope having a bendable part that allows a distal end portion to be bent to be larger than a proximal end portion.

JP2004-154545A describes an endoscope having a bendable part which is bendable with a plurality of curvature radii.

JP2000-342517A describes that in a bendable part structure of an endoscope configured by connecting a plurality of nodal rings along an axial direction of an insertion part, the plurality of nodal rings are connected such that an interval between adjacent nodal ring bodies gradually increases from a proximal end part side of the bendable part toward a distal end part side of the bendable part.

### SUMMARY OF THE INVENTION

In the technology of the present disclosure, an endoscope that can easily insert a distal end part to the back of a bronchus is provided.

An endoscope according to an aspect of the present disclosed technology comprises an insertion part including a distal end part and a bendable part provided on a proximal end side of the distal end part; and a bending operation part capable of performing a bending operation on the bendable part, in which the bendable part includes a first region and a second region between the first region and the distal end part, the first region is bent in one direction with a first curvature radius in accordance with an operation of the bending operation part, the second region is bent in the one direction with a second curvature radius less than the first curvature radius in accordance with an operation of the bending operation part, and a length of the second region in a longitudinal axis direction of the insertion part is equal to or less than a length of the first region in the longitudinal axis direction.

According to the technology of the present disclosure, it is possible to provide an endoscope in which a distal end part can be easily inserted to the back of a bronchus.

### BRIEF DESCRIPTION OF THE DRAWINGS

FIG. 1 is an overall view of an endoscope 1 of one aspect of the technology of the present disclosure.

FIG. 2 is a perspective view of a distal end hard part 34 including a part of a bendable part 32.

FIG. 3 is a perspective view of the distal end hard part 34 in a state where an angle rubber 36 shown in FIG. 2 is removed.

FIG. 4 is a perspective view schematically showing a main part of the bendable part 32 of the endoscope 1 shown in FIG. 1.

FIG. 5 is an enlarged perspective view of a first movable member 32A shown in FIG. 4.

FIG. 6 is an enlarged perspective view of a movable member 32B shown in FIG. 4.

FIG. 7 is a view schematically showing a state in which the bendable part 32 shown in FIG. 4 is viewed in a direction X.

FIG. 8 is a schematic view for describing a bent state of the bendable part 32 in a case where an angle lever 16 is rotated in an A1 direction from the state shown in FIG. 7.

FIG. 9 is a schematic view for describing a state in which the endoscope 1 is inserted into a bronchus and used.

FIG. 10 is a schematic view for describing a bent state of the bendable part 32 in a case where a rotation angle of the angle lever 16 is increased from the state shown in FIG. 8.

FIG. 11 is a schematic view for describing a state in which the endoscope 1 is inserted into the bronchus and used.

FIG. 12 is a schematic view for describing an example of a bent state of the bendable part 32 in a case where the angle lever 16 is rotated in a −A1 direction from the state shown in FIG. 7.

### DESCRIPTION OF THE PREFERRED EMBODIMENTS

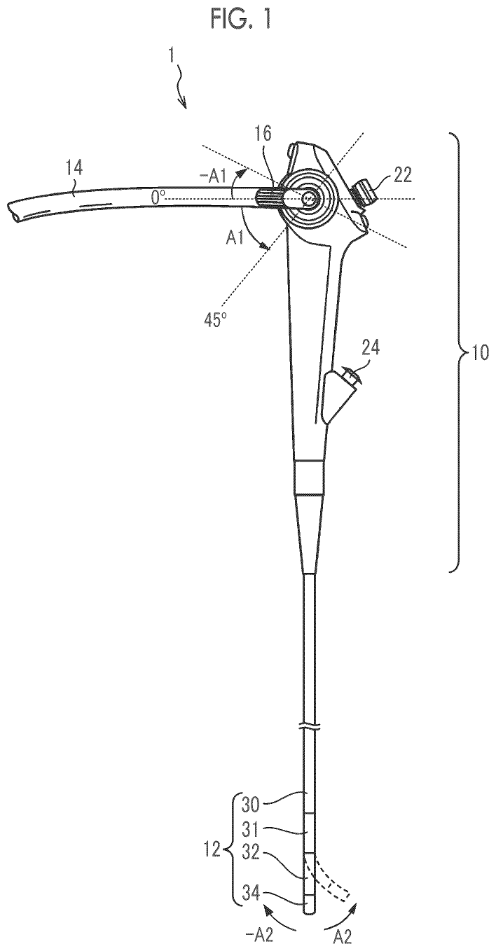

FIG. 1 is an overall view of an endoscope 1 according to an aspect of the technology of the present disclosure. As shown in FIG. 1, an endoscope 1 is used for collection of a cell tissue of a lesion part (may also be an observation part, an examination part, or a medical examination part). In the present embodiment, a bronchus will be described as an example of the part including the lesion part. For example, the bronchoscope is used for observation, examination, and the like of a portion of the bronchus where cancer is suspected.

The endoscope 1 is configured with an operating part 10 that is gripped by a practitioner to perform various operations, an insertion part 12 that is inserted into a body of a patient, and a universal cord 14. The endoscope 1 is connected to system constituent devices that configure an endoscope system, such as a processor device and a light source device (not shown), through the universal cord 14.

The operating part 10 is provided with various operation members that are operated by the practitioner. For example, an angle lever 16, a suction button 22, and the like of which the operations will be appropriately described below are provided.

The operating part 10 is provided with a treatment tool inlet port 24 through which a treatment tool is inserted into a treatment tool insertion channel (not shown) that is inserted into the insertion part 12.

The insertion part 12 extends from a distal end of the operating part 10 and is formed in a small-diameter elongated shape as a whole. The insertion part 12 is configured to be provided with a soft part 30, a passive bendable part 31, a bendable part 32, and a distal end hard part 34 which is a distal end part in order from a proximal end side to a distal end side.

The soft part 30 occupies most of the insertion part 12 from the proximal end side and has enough flexibility to be bent in any direction. In a case where the insertion part 12 is inserted into a body cavity, the soft part 30 is bent along an insertion path into the body cavity.

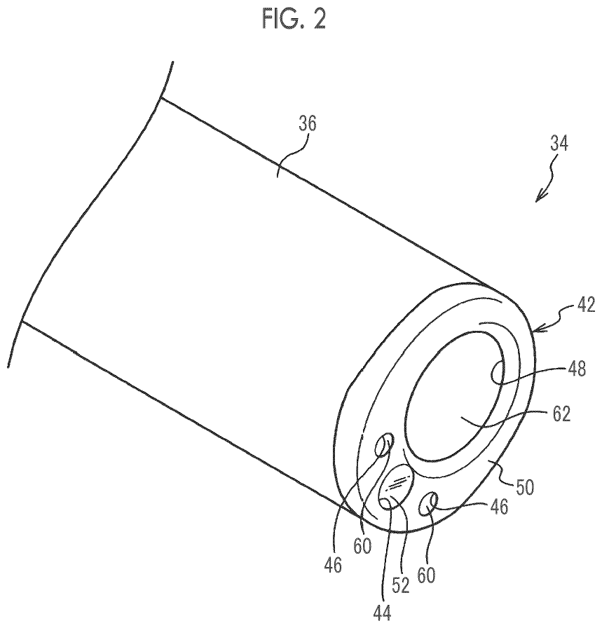

The passive bendable part 31 is composed of, for example, a metal spiral tube (a metal plate spirally wound) and a net (not shown), and the outer periphery of the passive bendable part 31 is further covered with a resin angle rubber 36 (see FIG. 2) together with a bendable part 32 described later. The passive bendable part 31 is configured to be more flexible than the soft part 30 so as to be able to be passively bent according to an external force, and bends along the insertion path into the body cavity in the same manner as the soft part 30 in a case where the insertion part 12 is inserted into the body cavity.

The bendable part 32 is bent in an up-down direction (an A2 direction and a-A2 direction opposite to the A2 direction) by rotationally operating the angle lever 16 of the operating part 10 in an A1 direction and a −A1 direction opposite to the A1 direction, and the distal end hard part 34 can be directed in a desired direction by bending the bendable part 32.

The angle lever 16 is configured to be rotated up to a rotation angle of 45° in the A1 direction with the state shown in FIG. 1 as a rotation angle of 0°, and the bendable part 32 is bent in the A2 direction (upward direction) by rotating the angle lever 16 in the A1 direction. The angle lever 16 is configured to be rotated up to an angle (for example, 25°) less than, for example, a rotation angle of 45° in the −A1 direction, and the bendable part 32 is bent in the −A2 direction (downward direction) by rotating the angle lever 16 in the −A1 direction. The angle lever 16 constitutes a bending operation part that can perform a bending operation of the bendable part 32. The A2 direction constitutes one direction.

The distal end hard part 34 comprises an observation optical system and an illumination optical system for capturing an observation image inside the body, and an outlet port 48 through which a treatment tool inserted from the treatment tool inlet port 24 is led out.

The universal cord 14 encompasses a signal cable connected to the observation optical system of the distal end hard part 34 and a light guide connected to the illumination optical system. A connector is provided in an end part (not shown) of the universal cord 14. The connector is connected to predetermined system constituent devices that configure the endoscope system, such as a processor device and a light source device. As a result, power, control signals, illumination light, and the like necessary for the operation of the endoscope 1 are supplied from the system constituent devices to the endoscope 1. In addition, conversely, the data of the observation image acquired by the observation optical system is transmitted from the endoscope 1 to the system constituent device. The observation image transmitted to the system constituent device is displayed on a monitor and can be observed by the practitioner or the like.

The configuration of the operating part 10 is not limited to the aspect shown in FIG. 1. Instead of the angle lever 16, an angle knob may be provided, or a pair of angle levers 16 or a pair of angle knobs may be provided, and the bending operation of the bendable part 32 may be performed in the up-down direction (A2 direction and −A2 direction) and a left-right direction orthogonal to the up-down direction by performing a rotation operation on the pair of angle levers 16 or the pair of angle knobs. An air/water supply button may be provided in the operating part 10, and gas, such as air, a liquid for cleaning, and the like may be supplied to the distal end hard part 34 by operating the air/water supply button.

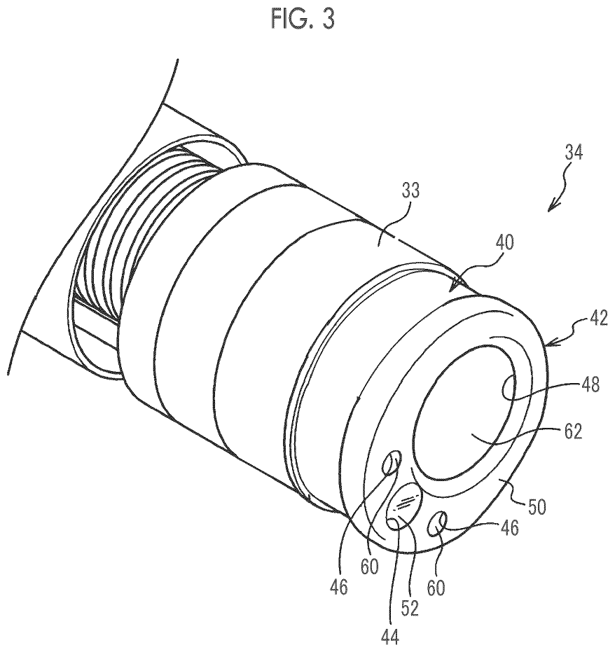

FIG. 2 is a perspective view of the distal end hard part 34 including a part of the bendable part 32. FIG. 3 is a perspective view of the distal end hard part 34 in a state in which the angle rubber 36 shown in FIG. 2 is removed.

As shown in FIGS. 2 and 3, the distal end hard part 34 has a distal end part body 40 and a cap 42 mounted on a distal end surface of the distal end part body 40. The distal end part body 40 is made of metal in order to reduce the diameter of the distal end hard part 34. The cap 42 is made of a resin or the like.

The angle rubber 36 shown in FIG. 2 extends to the proximal end side of the cap 42 to cover the distal end part body 40, and the distal end part of the angle rubber 36 is wound around a thread (not shown) and fixed to the distal end part body 40. The thread is fixed to the angle rubber 36 with an adhesive (not shown).

FIG. 3 also shows a distal end-side connecting member 33 made of metal, which is connected to the distal end part body 40. The distal end-side connecting member 33 is provided on the distal end side of the bendable part 32.

The cap 42 is provided with an opening 44 for an observation window, a pair of openings 46 for illumination windows, and an outlet 48. The opening 44, the pair of openings 46, and the outlet 48 are provided as through-holes in a front surface part 50 of the cap 42, respectively. In the front surface part 50, a region occupied by the outlet port 48 is the largest, the opening 44 is disposed on a lower side in the drawing of the outlet port 48, and the openings 46 are disposed on both sides of the opening 44.

An observation optical system composed of a lens barrel or the like holding an observation window (lens or protective glass) 52 and an imaging unit composed of an imaging element (not shown) or the like are provided at a position of the distal end part main body 40 facing the opening 44. In a case where the cap 42 is mounted on the distal end part body 40, the observation window 52 is exposed from the opening 44.

An illumination window (lens or protective glass) 60 of a light guide (not shown) is disposed at a position of the distal end part body 40 facing the pair of openings 46. In a case where the cap 42 is mounted on the distal end part body 40, the illumination windows 60 are exposed from the pair of openings 46, respectively. The light guide is connected to the light source device and supplies illumination light from the light source device to the illumination window 60.

A treatment tool insertion hole 62 is disposed at a position of the distal end part main body 40 facing the outlet port 48. In a case where the cap 42 is mounted on the distal end part body 40, the treatment tool insertion hole 62 communicates with the outlet port 48. As a result, the treatment tool inserted from the treatment tool inlet port 24 is led out to the outside from the outlet port 48 via the treatment tool insertion hole 62.

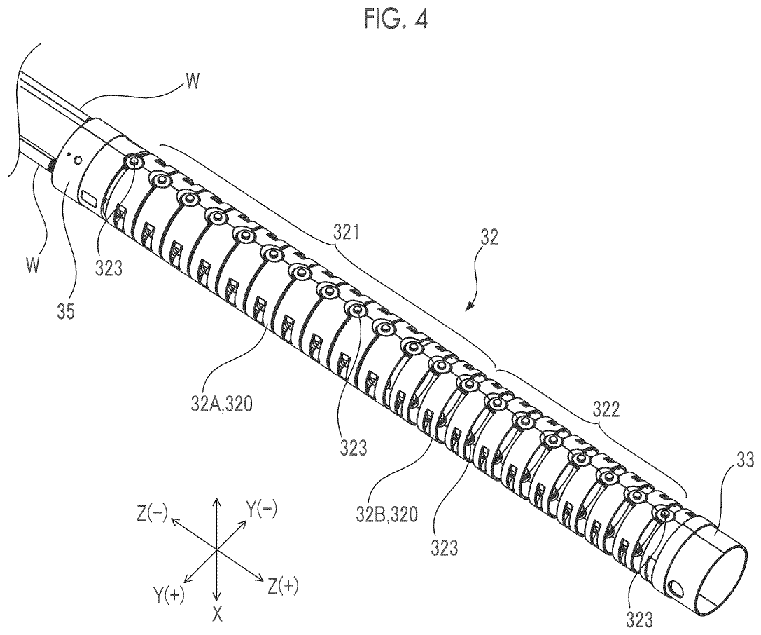

FIG. 4 is a perspective view schematically showing a main part of the bendable part 32 of the endoscope 1 shown in FIG. 1. A Z direction in the drawing is a direction parallel to a longitudinal axis of the insertion part 12. The Z direction constitutes a longitudinal axis direction. A Z(+) direction side, which is one side of the Z direction in the drawing, is a distal end side of the insertion part 12, and a Z(−) direction side is a proximal end side of the insertion part 12. A Y direction in the drawing is a direction perpendicular to a Z direction and corresponds to an A2 direction and a-A2 direction in FIG. 1. A Y(+) direction, which is one side of the Y direction, is the A2 direction in FIG. 1, and a Y(−) direction, which is the other side of the Y direction, is the −A2 direction in FIG. 1. An X direction in the drawing is a direction perpendicular to both the Z direction and the Y direction.

The bendable part 32 comprises a plurality of movable members 320 arranged in the Z direction, a proximal end-side connecting member 35 that connects the soft part 30 to a most proximal end side (Z(−) direction side) of the plurality of movable members 320, a distal end-side connecting member 33 that connects the distal end hard part 34 to a most distal end side (Z(+) direction side) of the plurality of movable members 320, and two wires W inserted into each of the movable members 320, the proximal end-side connecting member 35, and the distal end-side connecting member 33.

The bendable part 32 includes a first region 321 and a second region 322 between the first region 321 and the distal end hard part 34. The first region 321 and the second region 322 are configured to be bendable with different curvature radii. Specifically, the first region 321 is configured to be bendable with a first curvature radius, and the second region 322 is configured to be bendable with a second curvature radius less than the first curvature radius. That is, the bendable part 32 is configured to be bent more on the distal end side than on the proximal end side.

In the example of FIG. 4, a total of 19 movable members 320 are provided. The first region 321 is configured by including 12 movable members 320 arranged from the proximal end-side connecting member 35 side among the 19 movable members 320. The second region 322 is configured by including seven movable members 320 arranged from the distal end-side connecting member 33 side among the 19 movable members 320.

Each of the plurality of movable members 320 is formed of a cylindrical member having an axis extending in the Z direction. The plurality of movable members 320 include a first movable member 32A and a second movable member 32B having a width in the Z direction less than that of the first movable member 32A. Among the 19 movable members 320, the first 10 movable members 320 counted from the proximal end-side connecting member 35 side are defined as first movable members 32A, and the movable members 320 counted from the proximal end-side connecting member 35 side, starting from the eleventh onward, are defined as second movable members 32B.

The first region 321 is configured by including ten first movable members 32A and two second movable members 32B. The second region 322 is configured by including seven second movable members 32B. The first movable member 32A closest to the proximal end side of the first region 321 is rotationally movably connected to the tubular proximal end-side connecting member 35. The second movable member 32B closest to the distal end side in the second region 322 is rotationally movably connected to the tubular distal end-side connecting member 33.

Each of the two wires W passes through the soft part 30 and is connected to the angle lever 16. Each movable member 320, the proximal end-side connecting member 35, and the distal end-side connecting member 33 are made of, for example, metal, resin, or the like. The distal end of each wire W is anchored to the distal end-side connecting member 33.

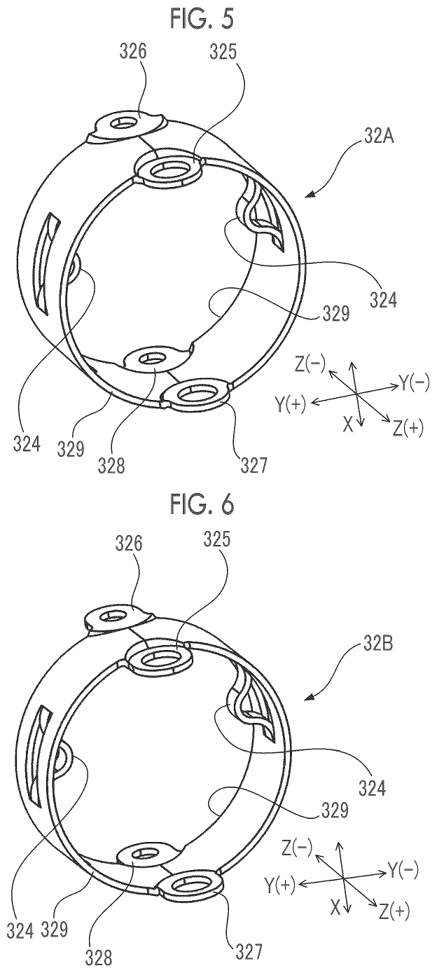

FIG. 5 is an enlarged perspective view of the first movable member 32A shown in FIG. 4. FIG. 6 is an enlarged perspective view of the second movable member 32B shown in FIG. 4.

An end surface 329 of the first movable member 32A on the Z(+) direction side and an end surface 329 of the first movable member 32A on the Z(−) direction side are each a flat surface perpendicular to the Z direction.

The first movable member 32A is provided with a connection piece 325 that protrudes from an end surface 329 on the Z(+) direction side to the Z(+) direction at one end part in the X direction, and a connection piece 326 that protrudes from an end surface 329 on the Z(−) direction side to the Z(−) direction at the one end part. The connection piece 325 and the connection piece 326 have a plate shape having a thickness in the X direction. Each of the connection piece 325 and the connection piece 326 is provided with a connecting hole that penetrates in the X direction.

The first movable member 32A is provided with a connection piece 327 that protrudes from an end surface 329 on the Z(+) direction side to the Z(+) direction at the other end part in the X direction, and a connection piece 328 that protrudes from an end surface 329 on the Z(−) direction side to the Z(−) direction at the other end part in the X direction. The connection piece 327 and the connection piece 328 have a plate shape having a thickness in the X direction. Each of the connection piece 327 and the connection piece 328 is provided with a connecting hole that penetrates in the X direction.

A pair of wire guides 324 into which two wires W are inserted is provided on an inner peripheral surface of the first movable member 32A in the Y direction so as to face each other between one end part and the other end part in the X direction.

As shown in FIG. 6, the second movable member 32B has the same configuration as the first movable member 32A, except that the width of the second movable member 32B in the Z direction (the distance between the end surface 329 on the Z(+) direction side and the end surface 329 on the Z(−) direction side) is less than that of the first movable member 32A.

As shown in FIG. 4, the connection piece 325 of each movable member 320 except for two movable members 320 closest to the proximal end side and the distal end side among all the movable members 320 is rotationally movably connected to the connection piece 326 of the movable member 320 adjacent to the distal end side by the connecting member 323, such as a rivet. The connection piece 326 of each movable member 320 is rotationally movably connected to the connection piece 325 of the movable member 320 adjacent to the proximal end side thereof by a connecting member, such as a rivet. The connection piece 327 of each movable member 320 is rotationally movably connected to the connection piece 328 of the movable member 320 adjacent to the distal end side thereof by a connecting member, such as a rivet. The connection piece 328 of each movable member 320 is rotationally movably connected to the connection piece 327 of the movable member 320 adjacent to the proximal end side thereof by a connecting member, such as a rivet.

The movable member 320 closest to the proximal end side among all the movable members 320 is rotatably connected to the proximal end-side connecting member 35 by a connecting member 323, such as a rivet, at the connection piece 326 and the connection piece 328. The movable member 320 closest to the distal end side among all the movable members 320 is rotatably connected to the distal end-side connecting member 33 by a connecting member 323, such as a rivet, at the connection piece 325 and the connection piece 327.

In the endoscope 1, the wire W inserted into the wire guide 324 on the Y(+) direction side of the wire guide 324 of the movable member 320 is pulled by the rotation operation of the angle lever 16 in the A1 direction, so that the bendable part 32 is bent in the Y(+) direction (A2 direction in FIG. 1). The wire W inserted into the wire guide 324 on the Y(−) direction side of the wire guide 324 of the movable member 320 is pulled by the rotation operation of the angle lever 16 in the −A1 direction, so that the bendable part 32 is bent in the Y(−) direction (the −A2 direction in FIG. 1).

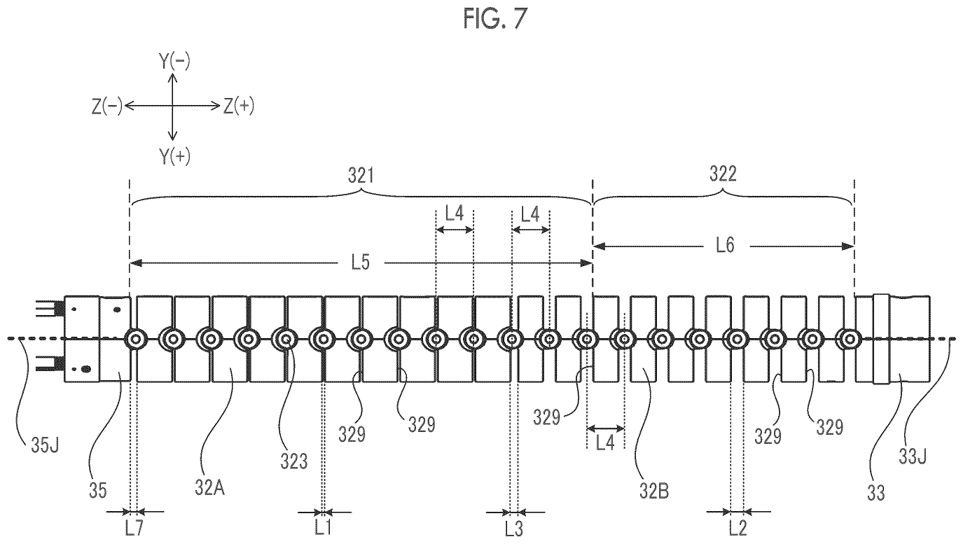

FIG. 7 is a diagram schematically showing a state in which the bendable part 32 shown in FIG. 4 is viewed in the direction X. As shown in FIG. 7, a gap is provided between end surfaces 329 of two adjacent first movable members 32A, and a length of the gap in the Z direction is a distance L1. A gap is provided between the end surfaces 329 of the two adjacent second movable members 32B, and a length of the gap in the Z direction is a distance L2. A gap is provided between the end surfaces 329 of the first movable member 32A that is the tenth movable member counted from the proximal end side and the second movable member 32B adjacent to the first movable member 32A, and a length of the gap in the Z direction is a distance L3. Intervals L4 in the Z direction between the connecting members 323 that connect the adjacent movable members 320 to be rotationally movable are uniform as a whole.

Since the interval L4 is uniform, the distance L2 is larger than the distance L1 due to a difference in width between the first movable member 32A and the second movable member 32B in the Z direction. The distance L3 has a magnitude obtained by adding half of the distance L2 and half of the distance L1.

A length of a gap between the proximal end-side connecting member 35 and the first movable member 32A adjacent to the proximal end-side connecting member 35 in the Z direction is a distance L7. It is preferable that the distance L7 is larger than the distance L1. Further, it is preferable that the distance L7 is less than the distance L2. It is preferable that the distance L7 is, for example, the same as the distance L3 or less than the distance L3. In this way, it is easy to increase the bending angle in a case where the first region 321 is maximally bent while suppressing the length of the first region 321 in the Z direction.

As shown in FIG. 7, a portion from a distal end-side end surface of the proximal end-side connecting member 35 to a proximal end-side end surface 329 of the second movable member 32B that is the seventh counted from the distal end-side connecting member 33 side constitutes the first region 321. In addition, a portion from the proximal end-side end surface of the distal end-side connecting member 33 to the proximal end-side end surface 329 of the second movable member 32B that is the seventh counted from the distal end-side connecting member 33 side constitutes the second region 322. It is preferable that a length L5 of the first region 321 in the Z direction is equal to or greater than a length L6 of the second region 322 in the Z direction.

In a case where the rotation angle of the angle lever 16 is 0°, as shown in FIG. 7, an axis 35J of the proximal end-side connecting member 35 and an axis 33J of the distal end-side connecting member 33 are arranged in a linear shape, and an angle formed by the axis 35J and the axis 33J is 0°. The axis 33J coincides with the axis of the distal end hard part 34. The axis 35J coincides with the axis of the soft part 30. In a case where the angle lever 16 is rotationally operated, the axis 33J of the distal end-side connecting member 33 is inclined with respect to the axis 35J of the proximal end-side connecting member 35, and an angle formed by the axis 35J and the axis 33J increases. An angle formed by the axis 35J and the axis 33J is defined as a bending angle of the bendable part 32. In the state shown in FIG. 4, the bending angle is 0°.

The bending angle of the bendable part 32 in a case where the angle lever 16 is rotated in the A1 direction to the maximum extent (in a case of a rotation angle of) 45° is also referred to as the maximum bending angle in the A2 direction. In consideration of enabling the distal end hard part 34 to be easily inserted into the bronchus having finely branched portions, the maximum bending angle in the A2 direction is preferably in a range of 165° or more and 195° or less, and more preferably 180°.

The bending angle of the bendable part 32 in a case where the angle lever 16 is rotated to the maximum extent in the −A1 direction is also referred to as the maximum bending angle in the −A2 direction. It is preferable that the maximum bending angle in the −A2 direction is less than the maximum bending angle in the A2 direction in consideration of the followability to the shape of the bronchus, the operability, and the optimization of the manufacturing cost. The maximum bending angle in the −A2 direction is, for example, preferably in a range of 90° or more and 150° or less, and more preferably 130°.

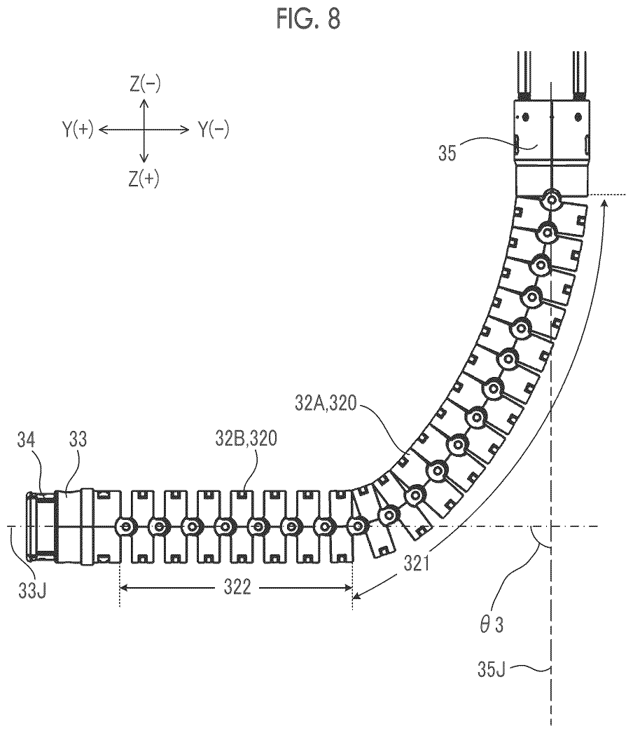

FIG. 8 is a schematic view for describing a bent state of the bendable part 32 in a case where the angle lever 16 is rotated in the A1 direction from the state shown in FIG. 7. The Y direction and the Z direction shown in FIG. 8 are shown as directions in a state in which the bendable part 32 is linearly extended up and down in the drawing (a state in which the bendable part 32 is not bent).

In a case where the angle lever 16 is rotated in the first range (range of rotation angle greater than 0° and 25° or less) in the A1 direction, as shown in FIG. 8, only the first region 321 out of the first region 321 and the second region 322 is bent in the Y(+) direction. On the other hand, the second region 322 maintains a state of extending along the axis 33J (synonymous with the axis of the distal end hard part 34).

FIG. 8 shows a state in which the angle lever 16 is rotationally operated in the first range and the bending angle is at its maximum (angle θ3) (state in which the rotation angle of the angle lever 16 is) 25°. The angle θ3 constitutes a first upper limit value of the bending angle of the first region 321 in a case where the first region 321 is bent in the A2 direction by the operation of the angle lever 16. Until the angle lever 16 reaches the rotation angle of 25°, the gap between the proximal end-side connecting member 35 and the adjacent movable member 320, the gap between the movable members 320 included in the first region 321, and the gap between the movable member 320 on the most distal end side in the first region 321 and the adjacent movable member 320 on the distal end side change in a direction in which the gaps gradually narrow, and the first region 321 is bent in the Y(+) direction.

On the other hand, while the first region 321 is bent, the gap between the movable members 320 included in the second region 322 and the gap between the distal end-side connecting member 33 and the adjacent movable member 320 are maintained in the state shown in FIG. 7. In the example shown in FIG. 8, the angle θ3 is set to 90°. As described above, until the bending angle reaches 0° to 90°, the orientation of the distal end hard part 34 can be changed while the second region 322 is kept in a linear shape.

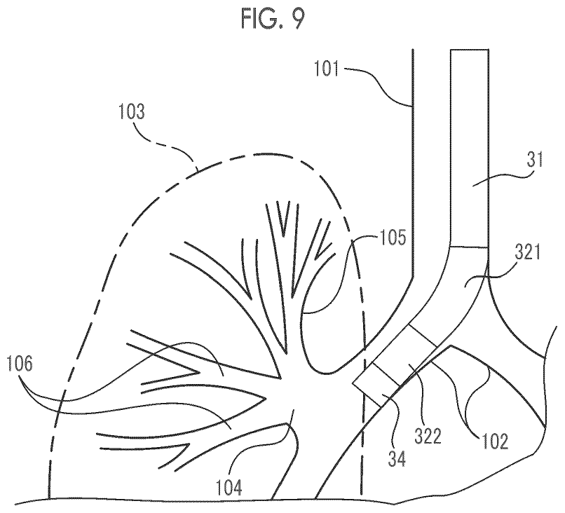

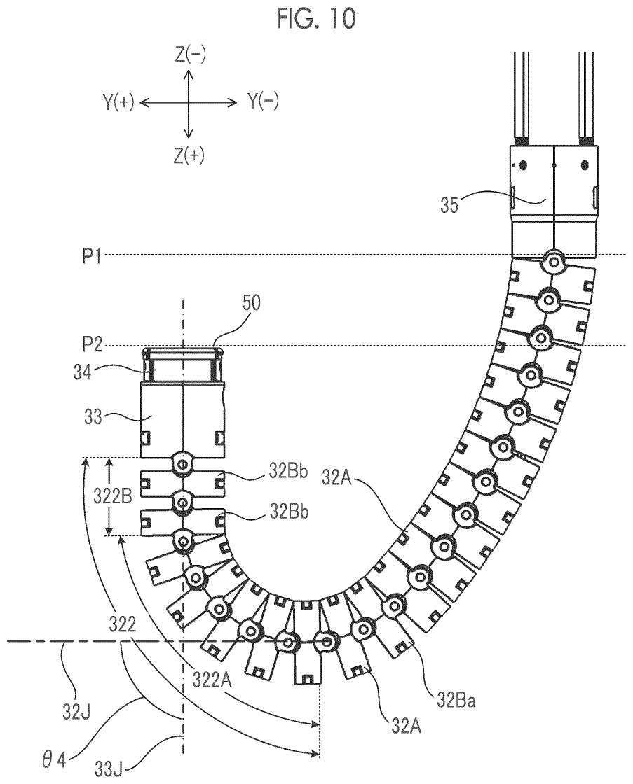

FIG. 9 is a schematic view for describing a state in which the endoscope 1 is inserted into the bronchus and used. FIG. 9 shows a trachea 101 of the subject, a lung 103 of the subject, a pair of main bronchi 102 that branch off from the trachea 101 to the left and right and to the lower side, and a bronchus 105 that branches off from the main bronchus 102 to the upper side via a superior lobar bronchus 104.

As described above, the endoscope 1 can bend the first region 321 with a large curvature radius while holding the second region 322 in a linear shape. As shown in FIG. 9, by curving the first region 321, the distal end hard part 34 can be smoothly inserted along a gentle and relatively long bending path from the trachea 101 to the main bronchus 102. For example, from the state shown in FIG. 9, the distal end hard part 34 is advanced to the back while the first region 321 is bent while the second region 322 is held in a linear shape, so that the distal end hard part 34 can be inserted into a bronchus 106 that branches from the superior lobar bronchus 104 to the back.

In addition, in consideration of the smooth insertion of the distal end hard part 34 into the bronchus, the angle θ3 shown in FIG. 8 is preferably 85° or more and 95° or less, and more preferably 90°.

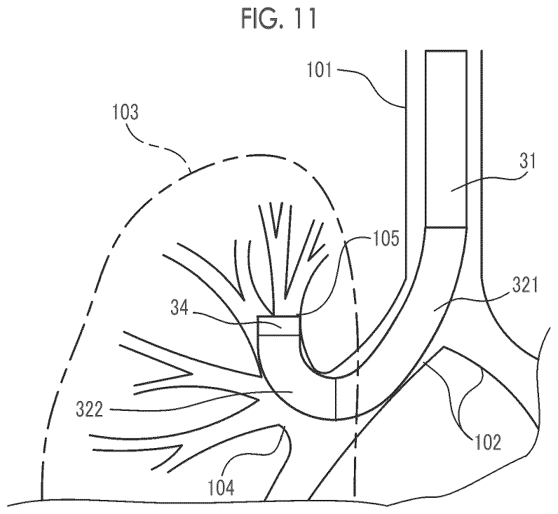

FIG. 10 is a schematic view for describing a bent state of the bendable part 32 in a case where the rotation angle of the angle lever 16 is increased from the state shown in FIG. 8. In a case where the angle lever 16 is rotated in a second range (a range of a rotation angle greater than 25° and 45° or less) exceeding the first range in the A1 direction, only the second region 322 out of the first region 321 and the second region 322 is bent as shown in FIG. 10. On the other hand, the first region 321 maintains the bent state shown in FIG. 8 (the bent state in a case where the rotation angle is 25° (the rotation angle of the end on the second range side in the first range)).

FIG. 10 shows a state in which the angle lever 16 is rotationally operated in the second range and the bending angle is maximized (state in which the rotation angle of the angle lever 16 is) 45°. In this state, an angle θ4 (defined as an angle formed by the axis 32J of the first movable member 32A on the most proximal end side of the second region 322 and the axis 33J of the distal end-side connecting member 33) indicating the bending angle of the second region 322 is 90°. Since the angle θ3 indicating the bending angle of the first region 321 shown in FIG. 8 is 90°, the bending angle of the bendable part 32 is 90°+90°=180° in the state shown in FIG. 10. The angle θ4 constitutes a second upper limit value of the bending angle of the second region 322 in a case where the second region 322 is bent in the A2 direction by the operation of the angle lever 16.

Until the angle lever 16 rotates from the rotation angle of 25° to the rotation angle of 45°, the gap between five movable members 320 excluding two movable members 320 on the distal end side (denoted by reference numeral 32Bb in FIG. 10) of the movable members 320 included in the second region 322 changes in a direction in which the gap gradually decreases, and a portion 322A (a part closer to the proximal end side with respect to the two movable members 32Bb) of the second region 322 is bent with a smaller curvature radius than the first region 321. As shown in FIG. 10, in a state where the rotation angle of the angle lever 16 is 45°, the axes of the two movable members 32Bb coincide with the axis 33J of the distal end-side connecting member 33. That is, even in a case where the angle lever 16 is rotated to the maximum extent in the A1 direction, the remaining portion 322B excluding the portion 322A in the second region 322 maintains a linear shape.

As described above, the other portion 322B of the second region 322 is configured such that the bending operation by the rotation of the angle lever 16 is not possible. In the present embodiment, the other portion 322B is prevented from being bent by regulating the rotatable range of the angle lever 16. However, the remaining portion 322B can be bent in each of the Y(+) direction and the Y(−) direction with the same curvature radius as the portion 322A in a case where an external force is applied. Since the other portion 322B has a linear shape, in a case where the treatment tool is led out from the outlet port 48, the lead-out can be smoothly performed. In the second region 322, the other portion 322B is not essential and can be omitted.

FIG. 11 is a schematic view for describing a state in which the endoscope 1 is inserted into the bronchus and used. In the clinical field, it may be necessary to observe or treat the periphery of the bronchus 105 that branches from the superior lobar bronchus 104 to the upper side.

In a case where the distal end hard part 34 is inserted into the bronchus 105, the distal end hard part 34 can be smoothly inserted along a gentle and relatively long bending path from the trachea 101 to the superior lobar bronchus 104 while bending the first region 321. Furthermore, since the second region 322 can be bent with a small curvature radius from a state in which the first region 321 is maximally bent, as shown in FIG. 11, the distal end hard part 34 can be easily inserted into the back of the bronchus 105 that branches at an acute angle with respect to the path from the trachea 101 to the superior lobar bronchus 104.

Even in a case where the distal end hard part 34 is inserted into the thin bronchus, the remaining portions 322B of the second region 322 can be passively bent, so that the insertion can be smoothly performed.

In addition, in a case where the second region 322 is bent from a state in which the first region 321 is bent to the maximum extent, the bent state of the first region 321 is maintained. Therefore, the direction of the distal end hard part 34 can be changed by bending the second region 322 in a state where the first region 321, for example, is brought into contact with the wall surface of the main bronchus 102 and is stabilized. In addition, since the passive bendable part 31 is provided in the endoscope 1, the passive bendable part 31 can be bent by being brought into contact with the bronchial wall surface in the states shown in FIGS. 9 and 11, and thus the orientation of the distal end hard part 34 can also be flexibly changed. In addition, only the bendable part 32 can be moved in two vertical directions, but by providing the passive bendable part 31, the direction of the distal end hard part 34 can be freely set to 360 degrees. As a result, it is possible to easily perform the examination of the bronchus.

As described above, in order to smoothly insert the distal end hard part 34 along the path from the trachea 101 to the main bronchus 102, from the main bronchus 102 to the superior lobar bronchus 104, and from the superior lobar bronchus 104 to the bronchus 105 on the upper side, the relationship between the length L5 of the first region 321 and the length L6 of the second region 322 shown in FIG. 7 is important.

In a case where the length L6 is larger than the length L5, it is not easy to handle the distal end hard part 34 in a narrow space from the superior lobar bronchus 104. On the other hand, by making the length L6 and the length L5 the same, the distal end hard part 34 can be inserted into the depth of the bronchus 105 while facilitating the handling of the distal end hard part 34 in a narrow space beyond the superior lobar bronchus 104.

In addition, in a case where the length L6 is set to be less than the length L5, it is possible to further facilitate the handling of the distal end hard part 34 in a narrow space beyond the superior lobar bronchus 104. However, in a case where the length L6 is excessively small, it is not easy to insert the distal end hard part 34 into the depth of the bronchus 105. In addition, in a case where the length L6 is excessively small, it is not easy to increase the curvature of the second region 322.

As a result of repeated verification in consideration of the above circumstances, it was found that, by setting the length L6 to 0.5 times or more and 1.0 times or less the length L5, it is possible to improve the operability of the insertion while inserting the distal end hard part 34 into the bronchus 105.

It is noted that, although an example is shown, it is preferable that the length L5 is set to 34.4 mm, the length L6 is set to 19.6 mm, the distance L1 is set to 0.2 mm, the distance L2 is set to 0.95 mm, the distance L3 is set to 0.575 mm, the interval L4 is set to 2.8 mm, the distance L7 is set to 0.4 mm, the outer diameter of the movable member 320 is set to 6.0 mm, and a curvature R of the second region 322 is set to R6.

In order to appropriately perform the examination or treatment of the bronchus, it is preferable that the angle θ4 shown in FIG. 10 is set to 85° or more and 95° or less. In this manner, as described above, the distal end hard part 34 can appropriately approach the bronchus that branches from the superior lobar bronchus 104 at an acute angle. Preferably, by setting the angle θ3 to the angle θ4 or more, the distal end hard part 34 can more appropriately approach the bronchus.

In the example of FIG. 10 in which the maximum bending angle in the A2 direction is set to 180°, in a state in which the bending angle of the bendable part 32 is 180°, a position P2 of the distal end surface (front surface part 50 of FIG. 3) of the distal end hard part 34 in the Z direction is configured to be positioned on the distal end side with respect to a position P1 of a proximal end edge of the first region 321.

With such a configuration, it is possible to further facilitate the handling of the distal end hard part 34 in a narrow space from the superior lobar bronchus 104. In addition, by configuring the position P2 to be in a range of more than ⅓ and ¼ or less of the first region 321 from the proximal end edge of the first region 321, the distal end hard part 34 can be appropriately inserted into the depth of the bronchus.

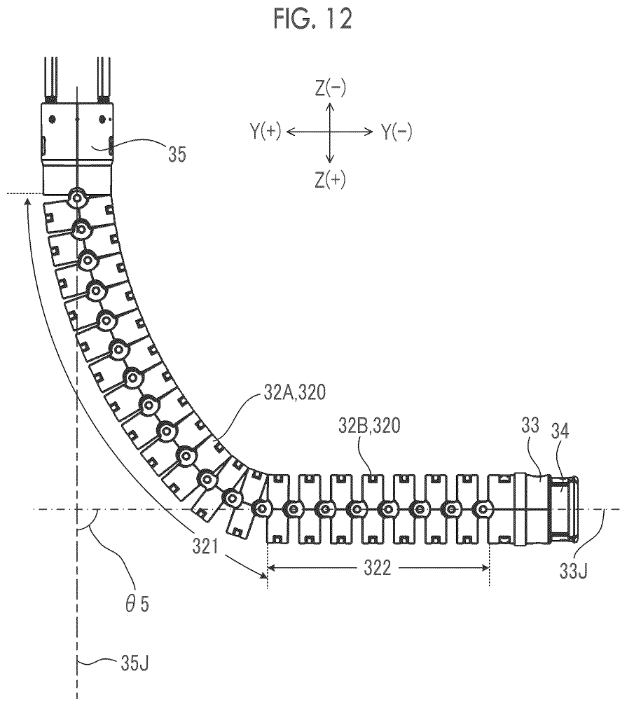

FIG. 12 is a schematic view for describing an example of a bent state of the bendable part 32 in a case where the angle lever 16 is rotated in the −A1 direction from the state shown in FIG. 7. The Y direction and the Z direction shown in FIG. 12 are shown as directions in a state in which the bendable part 32 is linearly extended up and down in the drawing (a state in which the bendable part 32 is not bent).

In a case where the angle lever 16 is rotated in a third range (a range of a rotation angle greater than 0° and 25° or less) in the −A1 direction, as shown in FIG. 12, only the first region 321 out of the first region 321 and the second region 322 is bent in the Y(−) direction. On the other hand, the second region 322 maintains a state of extending along the axis 33J (synonymous with the axis of the distal end hard part 34).

The bend state of the bendable part 32 in a case where the angle lever 16 is rotationally operated in the third range in the −A1 direction is the same as that in a case where the angle lever 16 is rotationally operated in the A1 direction in the first range, except that the bending direction of the bendable part 32 is the Y(−) direction. FIG. 12 shows a state in which the angle lever 16 is rotationally operated in the third range to have a maximum bending angle (angle θ5). The angle θ5 is 90° as an example. The angle θ5 is preferably the same as the angle θ3, but may be less than the angle θ3.

For example, as shown in FIG. 9, after the observation of the bronchus 105 by inserting the distal end hard part 34 into the main bronchus 102, and then returning the distal end hard part 34 to the position of the trachea 101, and then inserting the distal end hard part 34 into the bronchus below the superior lobar bronchus 104 of the main bronchus 102 on the right side in FIG. 9, it is sufficient that the maximum bending angle in the Y(−) direction is less than the maximum bending angle in the Y(+) direction.

In this way, by configuring the bending in the Y(−) direction as necessary and sufficient, it is possible to perform efficient operation depending on the part to be observed. In particular, in a case where the distal end hard part 34 is inserted into a narrow region such as a bronchus, it is preferable to bend the distal end hard part 34 as much as necessary, and from this viewpoint, it is preferable that the maximum bending angle in the Y(−) direction is less than the maximum bending angle in the Y(+) direction. In the example of FIG. 12, the maximum bending angle in the Y(−) direction is set to 90°, but the present invention is not limited thereto. For example, the second region 322 may be further bent from the state shown in FIG. 12 such that the maximum bending angle in the Y(−) direction is, for example, 130°.

As described above, at least the following matters are described in the present specification.

(1)

An endoscope including:
an insertion part including a distal end part and a bendable part provided on a proximal end side of the distal end part; and
a bending operation part capable of performing a bending operation on the bendable part,
in which the bendable part includes a first region and a second region between the first region and the distal end part,
the first region is bent in one direction with a first curvature radius in accordance with an operation of the bending operation part,
the second region is bent in the one direction with a second curvature radius less than the first curvature radius in accordance with an operation of the bending operation part, and
a length of the second region in a longitudinal axis direction of the insertion part is equal to or less than a length of the first region in the longitudinal axis direction.
   (2)

The endoscope according to (1),
in which the length of the second region in the longitudinal axis direction of the insertion part is 0.5 times or more the length of the first region in the longitudinal axis direction.
   (3)

The endoscope according to (2),
in which a first upper limit value of a bending angle of the first region in a case of bending in the one direction by the operation of the bending operation part is equal to or more than a second upper limit value of a bending angle of the second region in a case of bending in the one direction by the operation of the bending operation part.
   (4)

The endoscope according to (3),
in which the first upper limit value is 85° or more and 95° or less.
   (5)

The endoscope according to (4),
in which the second upper limit value is 85° or more and 95° or less.
   (6)

The endoscope according to (5),
in which an end part of the second region on a side of the distal end part is configured not to be subjected to the bending operation by the bending operation part.
   (7)

The endoscope according to (6),
in which the end part of the second region on the side of the distal end part is configured to be bendable by an external force.
   (8)

The endoscope according to any one of (1) to (7),
in which the bendable part is bendable in the one direction by a bending angle of at least 180° in accordance with an operation of the bending operation part, and
in a case where the bendable part is bent by 180° in the one direction, a distal end surface of the distal end part is positioned on a distal end side with respect to a proximal end edge of the first region.
   (9)

The endoscope according to (8),
in which, in a case where the bendable part is bent by 180° in the one direction, the distal end surface of the distal end part is positioned in a range of ⅓ or more and ¼ or less of the first region from the proximal end edge of the first region.
   (10)

The endoscope according to any one of (1) to (9),
in which, in a case where the bending operation part is operated in a first range, the first region is bent in the one direction, and
in a case where the bending operation part is operated in the first range, the second region is held in a state of extending along an axis of the distal end part.
   (11)

The endoscope according to (10),
in which, in a case where the bending operation part is operated in a second range exceeding the first range, at least a part of the second region is bent in the one direction.
   (12)

The endoscope according to (11),
in which, in a case where the bending operation part is operated in the second range, a state of bending of the first region when the bending operation part is operated to an end of the first range on a side of the second range is maintained.
   (13)

The endoscope according to any one of (1) to (12),
in which the insertion part has a soft part provided on a proximal end side of the bendable part and a passive bendable part provided between the soft part and the bendable part.
   (14)

An endoscope including:
an insertion part including a distal end part and a bendable part provided on a proximal end side of the distal end part; and
a bending operation part capable of performing a bending operation on the bendable part,
in which the bendable part includes a first region and a second region between the first region and the distal end part,
the first region is bent in one direction with a first curvature radius in accordance with an operation of the bending operation part,
the second region is bent in the one direction with a second curvature radius less than the first curvature radius in accordance with an operation of the bending operation part, and
an end part of the second region on a side of the distal end part is configured not to be subjected to the bending operation by the bending operation part.
   (15)

The endoscope according to (14),
in which the end part of the second region on the side of the distal end part is configured to be bendable by an external force.
   (16)

An endoscope including:
an insertion part including a distal end part and a bendable part provided on a proximal end side of the distal end part; and
a bending operation part capable of performing a bending operation on the bendable part,
in which the bendable part includes a first region and a second region between the first region and the distal end part,
the first region is bent in one direction with a first curvature radius in accordance with an operation of the bending operation part,
the second region is bent in the one direction with a second curvature radius less than the first curvature radius in accordance with an operation of the bending operation part,
the bendable part is bendable in the one direction by a bending angle of at least 180° in accordance with an operation of the bending operation part, and
in a case where the bendable part is bent by 180° in the one direction, a distal end surface of the distal end part is positioned on a distal end side relative to a proximal end edge of the first region.
   (17)

The endoscope according to (16),
in which, in a case where the bendable part is bent by 180° in the one direction, the distal end surface of the distal end part is positioned in a range of ⅓ or more and ¼ or less of the first region from the proximal end edge of the first region.

### EXPLANATION OF REFERENCES

1: endoscope
10: operating part
12: insertion part
14: universal cord
16: angle lever
22: suction button
24: treatment tool inlet port
30: soft part
31: passive bendable part
32: bendable part
32A: first movable member
32B, 32Bb: second movable member
320: movable member
33: distal end-side connecting member
33J, 35J: axis
34: distal end hard part
35: proximal end-side connecting member
36: angle rubber
40: distal end part body
42: cap
44, 46: opening
48: outlet port
50: front surface part
52: observation window
60: illumination window
62: treatment tool insertion hole
101: trachea
102: main bronchus
103: lung
104: superior lobar bronchus
105, 106: bronchus
321: first region
322: second region
322A: portion
322B: remaining portion
323: connecting member
324: wire guide
325, 326, 327, 328: connection piece
329: end surface
L1, L2, L3, L7: distance
P1, P2: position
L4: interval
L5, L6: length

What is claimed is:

1. An endoscope comprising:
an insertion part including a distal end part and a bendable part provided on a proximal end side of the distal end part; and
a bending operation part capable of performing a bending operation on the bendable part,
wherein the bendable part includes a first region and a second region between the first region and the distal end part,
the first region is bent in one direction with a first curvature radius in accordance with an operation of the bending operation part,
the second region is bent in the one direction with a second curvature radius less than the first curvature radius in accordance with an operation of the bending operation part, and
a length of the second region in a longitudinal axis direction of the insertion part is equal to or less than a length of the first region in the longitudinal axis direction.

2. The endoscope according to claim 1,
wherein the length of the second region in the longitudinal axis direction of the insertion part is 0.5 times or more the length of the first region in the longitudinal axis direction.

3. The endoscope according to claim 2,
wherein a first upper limit value of a bending angle of the first region in a case of bending in the one direction by the operation of the bending operation part is equal to or more than a second upper limit value of a bending angle of the second region in a case of bending in the one direction by the operation of the bending operation part.

4. The endoscope according to claim 3,
wherein the first upper limit value is 85° or more and 95° or less.

5. The endoscope according to claim 4,
wherein the second upper limit value is 85° or more and 95° or less.

6. The endoscope according to claim 5,
wherein an end part of the second region on a side of the distal end part is configured not to be subjected to the bending operation by the bending operation part.

7. The endoscope according to claim 6,
wherein the end part of the second region on the side of the distal end part is configured to be bendable by an external force.

8. The endoscope according to claim 1,
wherein the bendable part is bendable in the one direction by a bending angle of at least 180° in accordance with an operation of the bending operation part, and
in a case where the bendable part is bent by 180° in the one direction, a distal end surface of the distal end part is positioned on a distal end side with respect to a proximal end edge of the first region.

9. The endoscope according to claim 8,
wherein, in a case where the bendable part is bent by 180° in the one direction, the distal end surface of the distal end part is positioned in a range of ⅓ or more and ¼ or less of the first region from the proximal end edge of the first region.

10. The endoscope according to claim 9,
wherein, in a case where the bending operation part is operated in a first range, the first region is bent in the one direction, and
in a case where the bending operation part is operated in the first range, the second region is held in a state of extending along an axis of the distal end part.

11. The endoscope according to claim 10,
wherein, in a case where the bending operation part is operated in a second range exceeding the first range, at least a part of the second region is bent in the one direction.

12. The endoscope according to claim 11,
wherein, in a case where the bending operation part is operated in the second range, a state of bending of the first region when the bending operation part is operated to an end of the first range on a side of the second range is maintained.

13. The endoscope according to claim 12,
wherein the insertion part has a soft part provided on a proximal end side of the bendable part and a passive bendable part provided between the soft part and the bendable part.

14. An endoscope comprising:
an insertion part including a distal end part and a bendable part provided on a proximal end side of the distal end part; and
a bending operation part capable of performing a bending operation on the bendable part,
wherein the bendable part includes a first region and a second region between the first region and the distal end part,
the first region is bent in one direction with a first curvature radius in accordance with an operation of the bending operation part,
the second region is bent in the one direction with a second curvature radius less than the first curvature radius in accordance with an operation of the bending operation part, and
an end part of the second region on a side of the distal end part is configured not to be subjected to the bending operation by the bending operation part.

15. The endoscope according to claim 14,
wherein the end part of the second region on the side of the distal end part is configured to be bendable by an external force.

16. An endoscope comprising:
an insertion part including a distal end part and a bendable part provided on a proximal end side of the distal end part; and
a bending operation part capable of performing a bending operation on the bendable part,
wherein the bendable part includes a first region and a second region between the first region and the distal end part,
the first region is bent in one direction with a first curvature radius in accordance with an operation of the bending operation part,
the second region is bent in the one direction with a second curvature radius less than the first curvature radius in accordance with an operation of the bending operation part,
the bendable part is bendable in the one direction by a bending angle of at least 180° in accordance with an operation of the bending operation part, and
in a case where the bendable part is bent by 180° in the one direction, a distal end surface of the distal end part is positioned on a distal end side relative to a proximal end edge of the first region.

17. The endoscope according to claim 16,
wherein, in a case where the bendable part is bent by 180° in the one direction, the distal end surface of the distal end part is positioned in a range of ⅓ or more and ¼ or less of the first region from the proximal end edge of the first region.
